(19) United States
(12) Patent Application Publication
(10) Pub. No.: US 2024/0165233 A1
(43) Pub. Date: May 23, 2024

(54) COMBINED USE OF RARE-EARTH ELEMENT DOPED CALCIUM CARBONATE PARTICLES WITH ULTRASOUND FOR REDUCING LOCAL FAT

(71) Applicants: National Health Research Institutes, Zhunan Town (TW); National United University, Miaoli (TW); National Taiwan University, Taipei (TW)

(72) Inventors: Feng-Huei LIN, Miaoli County (TW); Gin-Shin CHEN, Miaoli County (TW); Ping-Yu SHIH, Miaoli County (TW); Ching-Yun CHEN, Miaoli County (TW); Li-Ze LIN, Miaoli County (TW); Che-Yung KUAN, Miaoli County (TW); Zhi-Yu CHEN, Miaoli County (TW); I-Hsuan YANG, Miaoli County (TW)

(73) Assignees: National Health Research Institutes, Zhunan Town (TW); National United University, Miaoli (TW); National Taiwan University, Taipei (TW)

(21) Appl. No.: 17/989,541

(22) Filed: Nov. 17, 2022

(51) Int. Cl. A61K 41/00; A61K 33/10; A61N 7/00

(52) U.S. Cl. CPC A61K 41/0028; A61K 33/10; A61N 7/00; A61N 2007/0008

(57) ABSTRACT

The present invention relates to a method for reducing localized fat deposits in a subject in need thereof in need thereof by topically treating the subject with rare-earth element doped calcium carbonate particles in combination with low-intensity ultrasound. The rare-earth element doped calcium carbonate particles have good biocompatibility and can increase reactive oxygen species (ROS) production and produce carbon dioxide (CO₂) and calcium ions (Ca²⁺) in the region of administration under the ultrasonic irradiation. The method of the present invention is effective in inducing adipocyte necrosis, inhibiting adipogenesis, and decreasing body weight and useful for body sculpture.

### TECHNOLOGY FIELD

The present invention relates to a method for reducing localized fat deposits in a subject in need thereof by topically treating the subject with rare-earth element doped calcium carbonate particles in combination with low-intensity ultrasound. The rare-earth element doped calcium carbonate particles have good biocompatibility and can increase reactive oxygen species (ROS) production and produce carbon dioxide (CO₂) and calcium ions (Ca²⁺) in the region of administration under the ultrasonic irradiation. The method of the present invention is effective in inducing adipocyte necrosis, inhibiting adipogenesis, and decreasing body weight and useful for body sculpture.

### BACKGROUND OF THE INVENTION

The excessive localized fat is a matter of great concern among subjects from current society. It affects image and body shape negatively; and results in dissatisfaction on individual (de Gusmao et al., 2020). Body sculpture refers to the use of either surgical or non-invasive techniques to modify the body for those who desire to fat reduction for specific problem areas, such as, abdomen, hips, thighs (Jewell et al., 2012). In 2018, the global market to body sculpture reached to US$6.1 billion; that might increase to $16.5 billion by 2025 (Michon, 2021).

Generally, the diet and exercise are the first suggestion to keep body shape in normal or as so-called attraction (Kordi et al., 2015). However, strict diets and intense daily exercise are difficult to maintain routinely for much longer time; that may result to fail (Mason et al., 2018). Liposuction is a surgical technique used to remove fat tissue to make people have a desired contour, which is among the top five cosmetic surgical procedures performed in United States of America (Jalian and Avram, 2012). Unfortunately, the side effects of liposuction include lidocaine toxicity, infections, numbness, fat embolism, or even death. Furthermore, the skin may locally appear contour irregularities, for instance, bumpy, wavy or withered due to uneven fat removal, poor skin elasticity and unusual healing (Mrad et al., 2019; Witte et al., 2020; O'Neill et al., 2021). Along with safety concerns, several noninvasive nonsurgical approaches have been developed for body sculpting, which have drawn more attentions in recent years (Jalian and Avram, 2012; Shek et al., 2014).

Ultrasound is one of powerful tools in medical image for diagnosis and very popular in rehabilitation as a therapeutic modality (Moreno-Moraga et al., 2007). Over the last decade, ultrasound has been developed to a commercial set in plastic surgery as physical lipolysis for body sculpture by specific ultrasonic parameters to break down fat tissue around the patients' waist.

As known, low-intensity ultrasound (for example, 0.5-17.5 W/cm²) would increase the inertial cavitation and then go through the bubble growth, finally to bubble implosion to generate the heat and stress to destroy the fat tissue for lipolysis (Zhou et al., 2017); however, the result of breaking down the fat tissue is not so promising, and it could only serve as the side treatment along with the liposuction (Tonucci et al., 2014). Alternatively, high-intensity focused ultrasound (HIFU) was developed to burn-down subcutaneous adipose tissue by high intensity (1000 W/cm²) with a special focusing plate to converge the ultrasonic waves to the intended ablation area. HIFU has been reported to induce rapid cell necrosis by the high energy and temperature generated from cavitation explosion; that might effectively dissipate adipose tissue. However, HIFU has been reported to burn the surface skin and charred surrounding tissues, causing a serious inflammatory response.

In summary, lipolysis by low-intensity ultrasonic provides a good method for non-invasive and low-risk body sculpturing, without requiring a recovery period. However, non-invasive ultrasonic lipolysis still has some potential shortcomings that need to be improved and to skip the shortages from HIFU.

### SUMMARY OF THE INVENTION

In this invention, it is unexpectedly found that rare-earth element doped calcium carbonate particles when combined with low-intensity ultrasound is effective in inhibiting adipogenesis without skin burning which can provide a mild and non-invasive treatment for body sculpture.

Therefore, in one aspect, the present invention provides a method for reducing localized fat deposits in a subject in need thereof, comprising administering to a region with localized fat deposits of the subject a composition comprising an effective amount of rare-earth element doped calcium carbonate particles and applying to the region of the subject an ultrasonic irradiation having parameters whereby the localized fat deposits in the region of the subject is reduced.

In some embodiments, the amount of calcium carbonate particles is effective in increasing reactive oxygen species (ROS) production and producing carbon dioxide (CO₂) and calcium ions (Ca²⁺) in the region of administration under the ultrasonic irradiation.

In some embodiments, the amount of the calcium carbonate particles is effective in inducing adipocyte necrosis, inhibiting adipogenesis, and decreasing body weight under the ultrasonic irradiation.

In some embodiments, the region where calcium carbonate particles are administered to include back, shoulders, neck, chest, abdomen, thighs, hips, legs, arms and face.

In some embodiments, the calcium carbonate particles are administered through parenteral route.

In some embodiments, the rare-earth element is selected from the group consisting of lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), yttrium (Y) and scandium (Sc).

In some embodiments, the rare-earth element is cerium (Ce) or europium (Eu).

In some embodiments, the rare-earth element is europium (Eu).

In some embodiments, the calcium carbonate particles are aggregated to have a diameter in the range of 1 to 4 μm.

In some embodiments, the ultrasonic irradiation has intensity of 0.1 to 10 W/cm².

In some embodiments, the ultrasonic irradiation has frequency of 0.1 to 10 MHz.

In some embodiments, the ultrasonic irradiation has a duty cycle ranging from 20% to 100%.

In some embodiments, the ultrasonic irradiation is applied 1 to 5 days per week, each day 5 to 600 seconds, for 1 to 6 weeks.

In some embodiments, the subject is overweight or obese.

In some embodiments, the subject is the subject is suffered from obesity.

In some embodiments, the subject has a BMI value of 18.5 to 24.9.

In some embodiments, the method of the present invention is performed for cosmetic purpose for improving body appearance (e.g. body sculpture).

In some embodiments, the method of the present invention is performed for therapeutic purpose for treating a disease, such as obesity.

In another aspect, the present invention provides use of rare-earth element doped calcium carbonate particles as described herein for manufacturing a medicament for reducing localized fat deposits in a subject in need thereof under ultrasonic irradiation.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 shows that the major mechanisms of the synthesized rare-earth element doped calcium carbonate particles with low-intensity ultrasound treatment to effectively remove away adipo-tissue and inhibit adipogenesis for body sculpture.

FIG. 2 shows the X-Ray Diffraction (XRD) pattern of CaCO₃:Ce and CaCO₃:Eu.

FIGS. 3A and 3B show the results of examination of the synthesized rare-earth element doped calcium carbonate particles under the transmission electron microscopy (TEM), including FIG. 3A showing the TEM photo of CaCO₃:Eu, and FIG. 3B showing the selected area electronic diffraction pattern of CaCO₃:Ce and CaCO₃:Eu.

FIG. 4 showing the scanning electron microscope (SEM) image of the synthesized CaCO₃:Ce and CaCO₃:Eu.

FIGS. 5A and 5B show the results of the chemical composition analysis by energy-dispersed spectrophotometer (EDS), including FIG. 5A showing the chemical composition of the synthesized CaCO₃:Ce, and FIG. 5B showing the chemical composition of the synthesized CaCO₃:Eu.

FIG. 6 shows the size distribution of CaCO₃:Ce and CaCO₃:Eu.

FIG. 7 shows the evaluation of cell viability of synthesized CeCO₃:Ce and CaCO₃:Eu, respectively, ***p<0.001.

FIG. 8 shows the increase reactive oxygen species (ROS) production of 3T3-L1 treated with CeCO₃:Ce and CaCO₃:Eu, respectively, under ultrasound irradiation ***p<0.001.

FIGS. 9A and 9B show the cell viability of CeCO₃:Ce and CaCO₃:Eu exposed to ultrasound stimulation, including FIG. 9 A showing the results evaluated by water-soluble tetrazolium salt (WST-1) assay, *p<0.05, and FIG. 9B showing the results of live/dead staining (scale bar: 100 μm).

FIG. 10 shows the weight change rate of SD rats treated with CaCO₃:Ce and CaCO₃:Eu, respectively and exposed to ultrasonic irradiation.

FIG. 11 shows the growth rate of waistline in SD rats after treated with CaCO₃:Ce and CaCO₃:Eu injection and ultrasound irradiation.

FIG. 12 shows the subcutaneous fat percentage in SD rats induced by CaCO₃:Ce and CaCO₃:Eu, respectively, activated by ultrasound. The subcutaneous fat percentage for the rats treated with the combination of the CaCO₃:Ce or CaCO₃:Eu injection and ultrasound stimulation at week 4 was 88.7 and 78.3%, respectively, compared with control group as 100%. Normal control, NC. Sham control, SC. Ultrasound, US.

FIG. 13 shows the results of measurement of body temperature on SD rats.

FIG. 14 shows the histological sectioning with H&E stain (scale bar: 100 μm).

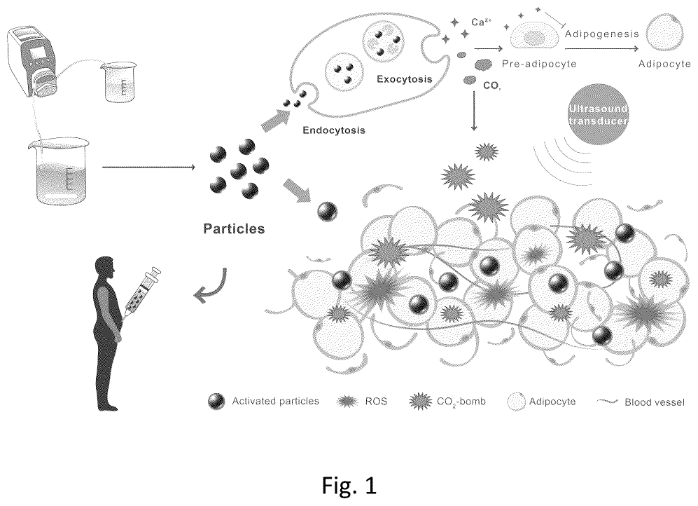

### DETAILED DESCRIPTION OF THE INVENTION

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a component” includes a plurality of such components and equivalents thereof known to those skilled in the art.

The term “comprise” or “comprising” is generally used in the sense of include/including which means permitting the presence of one or more features, ingredients or components. The term “comprise” or “comprising” encompasses the term “consists” or “consisting of.”

The term “about” as used herein means plus or minus 10% of the numerical value of the number with which it is being used. Therefore, about 1% means in the range of 0.9% to 1.1%.

1. Rare-Earth Element Doped Calcium Carbonate Particles

Calcium carbonate (CaCO₃) is the candidate material selected for the study due to its excellent biocompatibility and stability (Xiao et al., 2021). CaCO₃ is a biodegradable material that can decompose into carbon dioxide (CO₂) and calcium ions (Ca²⁺) in the acidic environment of endosome-lysosome complex. It is also one of materials with the property of sonoluminescence; where the particle could absorb the energy from the explosion of ultrasonic cavitation to generate heat to react with oxygen or biomolecules to induce reactive oxygen species (ROS) generation, and then convert into different free radicals to de-nature the proteins for cell necrosis (Jonnalagadda et al., 2021). In addition, CO₂ decomposed from CaCO₃ may serve as bomb to make cell damage under explosive stress, that could further kill the adipocyte (Yang et al., 2019). Ca² released from the breaking down of CaCO₃ at the acidic endosome-lysosome complex would increase the local calcium level around the adipose tissue; that might inhibit the differentiation of mesenchymal stem cells toward adipogenesis (Li et al., 2018).

In the present invention, a rare-earth element is dropped into the crystal lattice of calcium carbonate to provide rare-earth element doped calcium carbonate particles where the element partially replaces the Ca²⁺ in the lattice site of CaCO₃ in order to increase the sonoluminescent effect.

In some embodiments, the rare-earth element (represented by “X”) is selected from the group consisting of lanthanum (La), cerium (Ce), praseodymium (Pr), neodymium (Nd), samarium (Sm), europium (Eu), gadolinium (Gd), terbium (Tb), dysprosium (Dy), holmium (Ho), erbium (Er), thulium (Tm), ytterbium (Yb), lutetium (Lu), yttrium (Y) and scandium (Sc). Preferred examples of the rare-earth element as used in the present invention include cerium (Ce) and europium (Eu).

The rare-earth element doped calcium carbonate particles of the present invention can be prepared in a manner known in the art. For example, a green method is available for the preparation of the rare-earth element doped calcium carbonate particles of the present invention which is performed at relative-lower temperature without addition of organic solvent for environment friendly. Specifically, calcium nitrate (CaNO₃) and rare-earth element nitrate are dissolved in water to obtain a solution of calcium/rare-earth element nitrate; sodium carbonate (Na₂CO₃) is then added drop-by-drop into the solution of calcium/rare-earth element nitrate with stirring at room temperature for about 1.5 to 4 hours, and the resultant mixed solution is centrifugated to obtain precipitate of rare-earth element doped calcium carbonate (CaCO₃:X) which can be further washed by water and dried. The synthesized CaCO₃:X particles can be stored in desiccator for later use. The synthesized particles can be characterized using various methods known in the art. Specifically, x-ray diffractometer (XRD) can be used for the crystal structure identification of the synthesized particles. The morphology of the synthesized particles can be observed by scanning electron microscope (SEM). The semi-quantitative chemical composition of the synthesized particles can be examined and evaluated by energy-dispersed spectrophotometer (EDS) and inductively coupled plasma mass spectrometer (ICP-MS). The electronic diffraction pattern of the synthesized particles can be checked to know the crystal structure of the synthesized individual grain by transmission electron microscope (TEM). The particle size can be determined using a Zeta-sizer. Preferably, the synthesized particles per se have good biocompatibility. Cell viability assay can be used to evaluate that synthesized particles do not indue cytotoxicity to cells.

As used herein, the term “nanoparticle” is not limited to any particular shape, which should include various known shapes including, but not limited to, a sphere, a rod, a wire, a rhombohedrum, a cube and any other substantially spherical shape such as an ovoid. The term “nanoparticle” may have at least one dimension (e.g. a diameter) which is less than about 1 μm, preferably a particle having at least one dimension (e.g. a diameter) in the range from 5 to 900 nm, more preferably in the range from 10 to 500 nm, e.g. in the range from 10 to 300 nm. In some embodiments, nanoparticles may be aggregated to form clusters or microparticles, which are in a size larger than 1 μm, for example, in the range from 1 to 10 μm, e.g. in the range from 1 to 9 μm, 1 to 8 μm, 1 to 7 μm, 1 to 6 μm, 1 to 5 μm, 1 to 4 μm and 1 to 3 μm. The shape of clusters or microparticle can be scalenohedron or prism, for example.

In one particular embodiment, the rare-earth element is europium (Eu).

In one particular embodiment, the rare-earth element is cerium (Ce).

In one example, the Eu-doped calcium carbonate (Eu:CaCO₃) particles of the present invention have the characteristics as follows: “nailhead” or “dogtooth” spar of calcite crystals and a classic ring pattern of electronic diffraction; and aggregation into a particle in a size of appropriately 1 to 4 μm.

For the purpose of delivery and absorption, an effective amount of rare-earth element doped calcium carbonate particles as described herein as an active ingredient can be formulated with a physiologically acceptable carrier to form a composition in an appropriate form. Depending on the mode of administration, the composition of the present invention may contain about 0.1% to about 100% by weight of the active ingredient, wherein the percentage is calculated based on the total weight of the composition.

As used herein, “physiologically acceptable” means that the carrier is compatible with an active ingredient in the composition, and preferably can stabilize said active ingredient and is safe to the receiving individual. Said carrier may be a diluent, vehicle, excipient, or matrix to the active ingredient. Said carrier may be a diluent, vehicle, excipient, or matrix to the active ingredient. Some examples of suitable excipients include lactose, glucose, sucrose, sorbitol, mannitol, starch, Arabic gum, calcium phosphate, alginate, tragacanth gum, gelatin, calcium silicate, microcrystalline cellulose, polyvinylpyrrolidone, cellulose, sterile water, saline, syrup and methylcellulose. The composition may additionally contain lubricants, such as talc, magnesium stearate and mineral oil; wetting agents; emulsifiers and suspending agents; preservatives, such as methyl and propyl hydroxybenzoates; sweeteners; and flavoring agents. Typically, a composition comprising rare-earth element doped calcium carbonate particles as described herein as an active ingredient can be in a form of a solution such as an aqueous solution i.e., a saline solution or it can be provided in powder form. The composition may further contain pharmaceutically acceptable auxiliary substances as required to approximate physiological conditions, for example, pH adjusting and buffering agents, such as sodium acetate, sodium chloride, potassium chloride, calcium chloride, sodium lactate and the like. The form of the composition may be suspensions, lotions, solutions, sterilized injection fluid, and packaged powder. The composition of the present invention may be delivered via any physiologically acceptable route, such as parenteral (such as intramuscular, subcutaneous, and intraperitoneal) methods. In certain embodiments, the composition of the present invention is administered as a liquid injectable formulation which can be provided as a ready-to-use dosage form or as a reconstitutable stable powder.

In particular, the composition of the present invention may be administered parenterally, more particularly, topically administered through injection. Specifically, the topical administration includes topically injecting the composition of the present invention into the fat tissue (e.g. subcutaneous fat tissue) of a region of interest such as abdomen, back, calf, thigh, hip, under-chin, under-arm and under-eye.

The term “effective amount” as used herein refers to the amount of the active ingredient that imparts the desired biological effect in the individual or cell to be treated. The effective amount may vary depending on various reasons, such as the route and frequency of administration, the weight and species of the individual receiving the active ingredient, and the purpose of administration. Based on the content disclosed herein, the methods established, and their own experience, those skilled in the art can determine the dosage in each case.

II. Application of Ultrasound Irradiation

According to the present invention, rare-earth element doped calcium carbonate particles as described herein are used as a sonosensitizer to generate energy under application of ultrasound irradiation. Specifically, the sonication is applied locally to the area where the particles are administered to.

Ultrasound is a kind of mechanical wave that can transmit through different materials like fluids, soft tissues and solids. As used herein, sonosensitizers initiate a cytotoxic response in target tissues when exposed to ultrasonic energy. Sonosensitizers are activated by ultrasound and produce reactive oxygen species (ROS) that generate the cytotoxic effect. Sonodynamic treatment uses a sonosensitizer in combination with ultrasound which is non-invasive and capable of focusing on specific region of interest. Sonodynamic treatment involves activating preloaded, non-toxic compounds known as sonosensitizers using ultrasound. Such compounds may be specifically pre-absorbed or pre-incorporated into target cells and then produce cytotoxic effects upon activation by ultrasound.

As used herein, an ultrasound dose for an ultrasound treatment includes a series of ultrasound parameters, including, but not limited to, ultrasound intensity, ultrasound frequency, duty cycle, treatment time. These specific ultrasound parameters (ultrasound dose) vary and may depend on a variety of factors, including the age, body weight, general health, applying areas, and sex of the individual being treated, and also the type and severity of the particular condition (e.g. the level of localized fat deposits).

As used herein, ultrasound intensity means the ultrasonic power per unit area; particularly, the amount of energy (joules) flowing through a unit cross-sectional area (cm²) per a unit of time (seconds). As known in this art, in high-intensity focused ultrasound (HIFU) applications, the ultrasound intensity is usually greater than 100 W/cm², and even greater than 10,000 W/cm², which would cause rapid cell necrosis and also burned skin and charred surrounding tissues, resulting in serious inflammatory responses. In contrast, in the present invention, ultrasound is applied in a low intensity that is lower than the intensity used in HIFU which selectively induces adipocyte necrosis without unwanted damages such as skin burning and charred sounding tissue. In some embodiments, the ultrasound irradiation according to the present invention is applied at an intensity of no more than 100 W/cm², which can be 90 W/cm² or lower, 80 W/cm² or lower, 70 W/cm² or lower, 60 W/cm² or lower, 50 W/cm² or lower, 45 W/cm² or lower, 40 W/cm² or lower, 35 W/cm² or lower, 30 W/cm² or lower, 25 W/cm² or lower, 20 W/cm² or lower, 15 W/cm² or lower, 10 W/cm² or lower, 5 W/cm² or lower, 2.5 W/cm² or lower, 1 W/cm² or lower and 0.5 W/cm² or lower, while maintaining its selective capacity of inducing adipocyte necrosis without unwanted damages such as skin burning and charred sounding tissue. In some embodiments, the ultrasound intensity can be in a range of 0.1 to 100 W/cm², such as 0.1 to 90 W/cm², 0.1 to 80 W/cm², 0.1 to 70 W/cm², 0.1 to 60 W/cm², 0.1 to 50 W/cm², 0.1 to 40 W/cm², 0.1 to 30 W/cm², 0.1 to 20 W/cm², 0.1 to 10 W/cm², 0.1 to 5 W/cm², and 0.1 to 3 W/cm².

As used herein, ultrasound frequency is defined as the number of ultrasound waves per second and is measured in Hertz (cycles/second). Specifically, “khz” refers to kilohertz and a frequency magnitude of one thousand hertz; and “Mhz” refers to megahertz and a frequency magnitude of one million hertz. Ultrasound has a frequency higher than the upper human auditory limit of 20 KHz. The higher the frequency of ultrasound, the more rapid the absorption and the less distance propagation, while a low frequent ultrasound allows the acoustic energy to reach the deeper tissue. The present invention is based on the selection of a low intensity of ultrasound which can be used at any ultrasound frequency as long as the acoustic energy can reach to the region of interest where the CaCO₃:X particles as a sonosensitizer is administered, for example, a region with localized fat deposits, such as subcutaneous tissue. Preferably, the present invention uses the ultrasound frequency ranging from 0.1 to 10 MHz, for example, 0.1 MHz, 0.2 MHz, 0.5 MHz, 1 MHz, 2 MHz, 3 MHz, 4 MHz, 5 MHz, 6 MHz, 7 MHz, 8 MHz, 9 MHz and 10 MHz. In some embodiments, the ultrasound frequency can be in a range of 0.1 to 9 MHz, 0.1 to 8 MHz, 0.1 to 7 MHz, 0.1 to 6 MHz, 0.1 to 5 MHz, 0.1 to 4 MHz, 0.1 to 3 MHz, 0.1 to 2 MHz or 0.1 to 1 MHz.

In some embodiments, the ultrasound can be administered continuously (100% duty cycle). In other embodiments, the ultrasound can be administered intermittently (pulsed), using ON/OFF cycles which may range from 1% to 99%. In the case of using ON/OFF cycles, the term “duty cycle” is used to indicate the pulse duration divided by the pulse repetition period. In particular, duty cycle represents the fraction of time during which the ultrasound is working, for example, 10% DC means that the ultrasound is on for one (1) second and off for nine (9) seconds, and 50% DC means the ratio of on time to off time is 1:1. In some particular embodiments, the ultrasound is administered at a duty cycle ranging from 10% to 90%, such as 10%, 20%, 30%, 40%, 50%, 60%, 70%, 80% and 90%. In some particular embodiments, the ultrasound is administered at a duty cycle ranging from 20% to 90%, such as 20%, 30%, 40%, 50%, 60%, 70%, 80% and 90%. In some embodiments, the ultrasound is administered at a duty cycle ranging from 20% to 100%, such as 20%, 30%, 40%, 50%, 60%, 70%, 80% and 90% (intermittently (pulsed)) and 100% (continuously).

Additional ultrasound parameters include time of application, distance of transducer from the skin of the subject, number of treatment which can be determined by persons skilled in the art depending on the specific need and conditions.

The ultrasound treatment can be performed as a single treatment or as multiple courses of treatment. Persons skilled in the art can determine whether several successive courses of treatment are needed, based on the outcomes observed in the previous courses of treatment. In some embodiments, the application time of each separation application ranges from 5 seconds to 10 minutes, for example, 5 seconds to 9 minutes, 10 seconds to 8 minutes, 15 seconds to 7 minutes, 30 seconds to 6 minutes, 40 seconds to 5 minutes, 50 seconds to 4 minutes, and 1 to 3 minutes. In one example, the application time is about 90 seconds in each separate application. In one example, the application time is about 3 minutes in each separate application.

In some embodiments, the method of the present invention involves applying ultrasound irradiation at an intensity of 0.1 to 10 W/cm², a frequency of 0.1 to 10 MHz, a duty cycle ranging from 20% to 100% and at an application time of 5 to 600 seconds.

Preferably, the method of the present invention is non-invasive (no surgery is involved). Specifically, the method of the present invention involves placing an ultrasonic transducer in contact with or in close proximity to the surface of the region of a subject's body, optionally through a coupling medium, which is used to facilitate transmission of the ultrasound energy from the machine head to the tissues. Examples of a suitable coupling medium include gel, film, liquid, or ointment. In some embodiments, the ultrasound is applied at a transducer distance ranging from 0.5 cm to 5 cm from the subject.

In some embodiments, ultrasound treatment is performed immediately (such as within 5 minutes) after administration of the sonosensitizer particles as described herein. In some embodiments, ultrasound treatment may be applied after an incubation period (5 minutes or more) following administration of the sonosensitizer particles. In some embodiments, the incubation period is 5 to 30 minutes. In some embodiments, the incubation period is longer than 30 minutes, for examples 1 to 4 hours.

The ultrasound treatment can be performed in several successive courses as needed. In some particular embodiments, ultrasound irradiation is applied 1 to 5 days (such as 1 day, 2 days, 3 days 4 days or 5 days, consecutively or not) per week for 1 to 6 weeks or more (such as 1 week, 2 weeks, 3 weeks, 4 weeks, 5 weeks or 6 weeks or more).

III. Combined Use of Rare-Earth Element Doped Calcium Carbonate Particles with Ultrasound Irradiation

The present invention provides a method of combined treatment using rare-earth element doped calcium carbonate particles with ultrasound irradiation. The method of the present invention is useful for reducing localized fat deposits in a subject in need thereof.

As used herein, the term “treating” or “treatment” refers to an approach for obtaining beneficial or desired results. Particularly, the approach may include administration of sonosensitizer particles or a composition thereof as used herein in the form of a medicament to a subject in combination with sonication irradiation for the purpose of reducing localized fat deposits for cosmetic body sculpture. “Treating” and “treatment” can also include alleviation or amelioration of one or more symptoms or conditions, stabilized (i.e. not worsening) state of disease, diminishment of extent of disease, amelioration or palliation of the disease state.

As used herein, a subject in need according to the present invention includes humans and non-human mammals. Non-human mammals include, but are not limited to, companion animals such as cats, dogs and the like, and farm animals such as cows, horses, sheep, goats, pigs and the like. A subject can be a “healthy subject”. A healthy subject may be a subject not affected by a disease or disorder e.g. obesity. The World Health Organization uses the Body Mass Index (BMI) to measure the degree of obesity. The calculation formula is weight (kg) divided by the square of height (meters). According to the definition of the World Health Organization, normal individuals have a BMI value of 18.5 to 24.9, a BMI value greater than 25.0 is considered overweight, and a BMI value greater than 30.0 is considered obese. Among them, a BMI value of 30-34.9 is the first degree of obesity, and a BMI value of 35-39.9 is the second degree of obesity, and a BMI value of 40 or more is the third degree of obesity.

According to the present invention, combined treatment using rare-earth element doped calcium carbonate particles with ultrasound irradiation is used for reducing localized fat deposits in a subject in need thereof. In some embodiments, the combined treatment of the present invention is effective in slowing down the growing rate of localized fat deposits. In some examples, the combined treatment of the present invention reduces the amount (weight) of subcutaneous fat of a subject receiving the combined treatment by about 1 to 50% (e.g. 1 to 40%, 1 to 30%, 1 to 20% or 1 to 10%), compared with the reference the amount (weight) of subcutaneous fat of a control subject who does not receive the combined treatment.

In some embodiments, the method of the present invention is performed for cosmetic purpose for improving body appearance.

In some embodiments, the method of the present invention is performed for therapeutic purpose for treating a disease, such as obesity.

In some embodiments, the method of the present invention is applied to an overweight or obese subject.

In some embodiments, the method of the present invention is applied to a normal subject (BMI 18.5 to 24.9).

In some embodiments, the method of the present invention is applied to a subject suffering from obesity.

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### Examples

Body sculpture is a common method to remove excessive fat. The diet and exercise are the first suggestion to keep body shape; however, those are difficult to keep adherence. Ultrasound has been developed for fat ablation; however, it could only serve as the side treatment along with liposuction. In the study, a sonosensitizer of rare-earth element doped calcium carbonate particles (cerium-doped calcium carbonate (CaCO₃:Ce) or europium-doped calcium carbonate (CaCO₃:Eu)) would be synthesized by an eco-method and combined with low-intensity ultrasound for lipolysis. The crystal structure of the synthesized particles was identified by x-ray diffractometer (XRD). The morphology of the synthesized particles was analyzed by scanning electron microscope (SEM). The chemical composition of CaCO₃:Eu was evaluated by energy-dispersed spectrophotometer (EDS) and inductively coupled plasma mass spectrometer (ICP-MS). The electronic diffraction pattern was to further check crystal structure of the synthesized individual grain by transmission electron microscope (TEM). The particle size was determined by Zeta-sizer. Water-soluble tetrazolium salt (WST-1) were used to evaluate the cell viability. Chloromethyl-2′,7′-dichlorofluorescein diacetate (CM-H₂DCFDA) and live/dead stain were used to evaluate feasibility in vitro. SD-rat was used to evaluate the safety and efficacy in vivo. The results showed that the synthesized particles had good biocompatibility and could produce ROS after treated with low-intensity ultrasound. After 4-week, the synthesized particles exposed to ultrasound irradiation on SD rats could significantly decrease body weight, waistline, and subcutaneous adipose tissue. We believe that ROS from sonoluminescence, CO₂-bomb and locally increasing Ca²⁺ level would be three major mechanisms to remove away adipo-tissue and inhibit adipogenesis. We could say that the combination of the rare-earth element doped calcium carbonate particles and low-intensity ultrasound would be a non-invasive treatment for the body sculpture

1. Material and Methods

1.1 Cerium or Europium Doped Calcium Carbonate Preparation

CaCO₃:Eu was synthesized by an innovative method at room temperature without organic solvent addition for environment friendly. The process was briefly described as follows. Firstly, 1.18 g of calcium nitrate and 0.223 g of europium nitrate was dissolved in 50 mL of ddH₂O. Then, 50 mL of 0.1M sodium carbonate was added drop-by-drop into the previously prepared calcium/europium nitrate solution by peristaltic pump at 5.0 rpm and stirred by magnetic stirrer at room temperature for 3 hours, and the solution was centrifugated at 1300 rpm for 20 minutes (5500, Kubota, Japan). The precipitate was washed by ddH₂O for three times, and dried overnight in a freeze dryer (FDU-1100, EYELA, Japan) to obtain CaCO₃:Eu. The synthesized particles were stored in desiccator for later use. Cerium-doped calcium carbonate (CaCO₃:Ce) was prepared in a similar manner.

1.2 the Crystal Structure Identification

The crystal structure of the synthesized particles was identified by XRD (MiniFlex II, Rigaku, Japan) with Copper Ku-II radiation at 30 kV and 15 mA at a scan rate of 4°/minute from 200 to 60°. The sample was passed 230 mesh and pressed onto a sample holder with an area of 2 cm×2 cm.

1.3 the Morphological Examination and Grain Size Evaluation Under SEM

The morphology and grain size of the synthesized particles were examined and observed by a SEM (Hitachi™-1000, Japan). The sample were mounted on an aluminum-made SEM sample stage and then coated with a platinum film by a sputtering PVD. The sample edge was spotted with silver gel to prevent from undesired discharge to result in a blurry image.

1.4 the Analysis of Morphology and Electronic Diffraction Pattern by TEM

The morphology and electronic diffraction pattern of the developed particles were observed and analyzed by TEM (Tecnai G2 F20, FEI, USA). 5 mg of the particles were dispersed in 10 mL ddH₂O and homogenized by ultrasonic vibration for 15 minutes. 20 μL of the dispersed and homogenized particles were dropped on the carbon-coated copper mesh, and dried at room temperature in a petri-dish with lid covered to prevent from pollution from air. The accelerated voltage was 200 kV. The electronic diffraction pattern was obtained by selected area diffraction mode (SAD-mode).

1.5 Chemical Composition Analysis

The chemical composition of the material was analyzed by an EDS (JSM-5600, JEOL, Japan). The sample preparation was similar to process of the sample for SEM, but coated with a pyrolytic carbon rather than platinum film. The energy of the accelerated x-ray beam was 20 kV. The chemical composition of sample was further confirmed by an inductively coupled plasma mass spectrometer (ICP-MS, NexION 2000, PerkinElmer, USA). In brief, 20 mg of sample was dissolved in 200 μL of pure nitric acid (438073, Sigma, USA), and added with ddH₂O to 10 mL. The simple was diluted (1:10000) with ddH₂O and performed by ICP-MS with kinetic energy discrimination (KED) mode.

1.6 the Analysis of Particle Size Distribution

The particle size distribution of the synthesized particles was analyzed by using a Zeta-sizer (Nano ZS, Malvern, UK). The sample was firstly suspended in ddH₂O and homogenized by an ultrasonic vibration. The homogenized suspension was placed in a Zeta-sizer cell and then measured using Dynamic Light Scattering (DLS) at room temperature.

1.7 In Vitro Study

1.7.1 Evaluation of Cell Viability

The cell viability was evaluated by WST-1 on L-929 cell (RM60091, Bioresource Collection and Research Center, Taiwan); that would be in terms of in-vitro cytotoxicity based on the guideline of ISO 10993-5.

Briefly, L-929 cells were cultured in α-MEM (11900-024, Gibco, USA) supplemented with 10% fetal bovine serum (FBS, A31606-02, Hyclone, USA) and 1% of 100× antibiotic-antimycotic (Anti-anti, 15240-062, Gibco, USA); and then seeded to a 96-well culture plate with a cell density of 1×10⁴ per well and cultured at 37° C. under 5% CO₂ for 24 hours.

The culture medium would be used as the extraction vehicle to prepare sample extracted solution. 0.2 g of developed particles, aluminum oxide (11028, Sigma, USA) and polyurethane film containing 0.1% zinc diethyldithiocarbamate (ZDEC, RM-A, Hatano Research Institute, Food and Drug Safety Center, Japan) were immersed in 1 mL of culture medium, individually, at 37° C. under 5% CO₂ for 24 hours. The extracted solutions would be separately cultured with previous seeded cells and daily refreshed to evaluate cell viability; those would be named and abbreviated as experimental group (CaCO₃:Ce or CaCO₃:Eu), negative control (N-control) and positive control (P-control), respectively. The result of L-929 cells cultured with medium were the control group abbreviated as Control.

After one-day incubation, the medium was removed and then added in 90 μL culture medium and 10 μL WST-1 reagent (11644807001, Roche, USA); that was reacted at 37° C. under 5% CO₂ for 1 hour in dark. The culture plate was mounted on ELISA reader (VersaMax™, Molecular Devices, Canada); where the absorbance at the wavelength of 450 nm was recorded to evaluate the cell viability (Hsiao et al., 2019).

1.7.2 3T3-L1 Culture and Differentiation

Briefly, 3T3-L1 pre-adipocytes cell line (60159, Bioresource Collection and Research Center, Taiwan) was seeded to a 12-well culture plate with a cell density of 1×10⁴ per well and cultured at 37° C. under 5% CO₂ in Dulbecco Modified Eagle Medium (DMEM, high glucose, 12800-017, Gibco, USA) supplemented with 10% calf bovine serum (16170-078, Gibco, USA) and 1% of 100× Anti-anti. After confluence, it were further cultured in starvation condition for 2 days to keep cells in the status of G₀/G₁ phase at least 85% in all population (Cao et al., 2012). The confluent 3T3-L1 cells were cultured in an adipo-differentiated medium to convert cells into adipocytes; where the adipo-differentiated medium was DMEM supplemented with 10% FBS, 1% of 100× Anti-anti, 1 mM dexamethasone (D4902, Sigma, USA), 0.2M indomethacin (I7378, Sigma, USA), 0.1% insulin and 0.25M 3-Isobutyl-1-methylxanthine (IBMX, 15879, Sigma, USA)). The adipocytes were cultured in DMEM supplemented 10% FBS and 1% of 100× Anti-anti; and medium was refreshed every 3 days, until the oil droplets were observed by a fluorescence microscope (TS-100, Nikon, Japan) stained with Nile red (N1142, Invitrogen, USA) (Park et al., 2017).

1.7.3 ROS Generation

The ROS generation of adipocytes, induced by synthesized particles (CaCO₃:Ce or CaCO₃:Eu) and exposed to low-intensity ultrasound, was measured by CM-H₂DCFDA (C6827, Invitrogen, USA).

In brief, 3T3-L1 cells were seeded into 96-well culture plate with a density of 1×10⁴ cells per well and differentiated to adipocyte as described in section 1.7.2. 100 μL of 0.75 mg/mL particles in culture medium was added into each well and further cultured for 4 hours, and then exposed to low-intensity ultrasound from the bottom of the culture plate in degassed water by an ultrasound transducer with a diameter of 2.0 cm. The distance between ultrasound transducer and the bottom of the cell culture plate was around 5 mm. The ultrasound irradiation was conducted with a function generator (33521A, Agilent, USA) at a resonant frequency of 1.0 MHz and a duty cycle of 50%. A power amplify was used to generate a square wave with a negative pressure of 0.33 MPa and intensity of 1.8 W/cm² for 90 seconds (Yang et al., 2020). It was further cultured for 1 hour in the incubator. The medium was removed and the cells were stained with 25 μM CM-H₂DCFDA at room temperature for 45 minutes. The fluorescence was excited at the wavelength of 493 nm; and the intensity of emission light was measured by a multi-label plate reader (EnSpire, PerkinElmer, USA) at the wavelength of 523 nm that was the ROS concentration.

The experiment was divided into 4 groups and abbreviated in brace as follows: the cells were cultured in medium, (1) without particles addition and no ultrasound applied on (Control); (2) applied with low-intensity ultrasound without particles addition (Ultrasound); (3) with particles addition but no expose to low-intensity ultrasound (CaCO₃:Ce or CaCO₃:Eu); (4) with particles addition and expose to low-intensity ultrasound (US-CaCO₃:Ce or US-CaCO₃:Eu).

1.7.4 The In Vitro Screening of Adipocyte Treated with Synthesized Particles and Low-Intensity Ultrasound by WST-1 Assay and Live/Dead Stain

The cell viability and cytotoxicity of adipocyte, treated with synthesized particles (CaCO₃:Ce or CaCO₃:Eu) and exposed to low-intensity ultrasound, were evaluated by WST-1 assay and live/dead stain, respectively. The experiments were used as first screening in-vitro, trying to know the possibility of body sculpture in vivo once adipo-tissue treated with developed particles and followed by low-intensity ultrasound irradiation.

In brief, 3T3-L1 cells were seeded on 12-well culture plate with a density of 6×10⁴ cells per well and then differentiated into adipocyte. 0.75 mg/mL particles was added into each well and further cultured for 4 hours, and then exposed to low-intensity ultrasound. It was further cultured for 1 hour in the incubator. The medium was removed and then added in 900 μL culture medium and 100 μL WST-1 reagent; that was reacted at 37° C. under 5% CO₂ for 1 hour in dark. The culture plate was mounted on ELISA reader (EnSpire, PerkinElmer, USA); where the absorbance at the wavelength of 450 nm was recorded to evaluate the cell viability.

In the live/dead staining, the staining solution was prepared as follows; in which 50 μL of calcein AM (Ex/Em: 494/517 nm, C1430, Invitrogen, USA) and 16.5 μL of propidium iodide (PI, Ex/Em: 536/617 nm, P1304MP, Invitrogen, USA) reagents were well-mixed in phosphate buffered saline (PBS) and then added PBS to 5 mL, at pH 7.4. As previous description, the adipocytes were treated by developed particles and low-intensity ultrasound. After further cultured for 1 hour, the medium was removed and added in 400 μL of staining solution, reacted for 15 minutes at room temperature in dark. The culture plate was mounted on fluorescence microscope (TS100, Nikon, Japan), with which the living cells and dead cells would be labelled by calcein AM in green color and propidium iodide in red, respectively, under the proper excitation light.

1.8 In Vivo Study

1.8.1 Experimental Animals and Surgical Procedure

Sprague Dawley rat age 10-week old, 325 g body weight in average and male in gender was used in the study. The rats were purchased from BioLASCO, Taiwan, and delivered to Laboratory Animal Center, National Health Research Institutes, Taiwan, 7 days before the experiment started to accommodate the environment. One cage for one rat was conducted to all the experimental period with controlled temperature and humidity of 22° C. and 55%, respectively, by light turn-off and turn-on alternatively every 12 hours. The study protocol was approved by the Institutional Animal Care and Use Committee of the National Health Research Institutes (NHRI-IACUC-108012).

2.5 mg of CaCO₃:Ce or 3.75 mg of CaCO₃:Eu was mixed within 1 mL of normal saline. The 100 μL of mixture was injected into the fat tissue of abdomen area on the SD rats once a week for 4 weeks. The low-intensity ultrasound was applied on the area where particles was injected; and treated consecutively 3 days every week for 4 weeks, each day 90 seconds. The low-intensity ultrasound was generated by a function generator at a resonant frequency of 1.0 MHz, a duty cycle of 50%, a square wave with a negative pressure of 0.33 MPa and intensity of 1.8 W/cm².

The study was divided into 3 groups that was described and abbreviated as follows: (1) the rats without any treatment were categorized to Control Group (Control); (2) the rat received injection on abdomen fat tissue once a week by 100 μL normal saline was Sham Control; (3) the rat injected with particles once a week and received ultrasound treatment consecutively 3 days every week was the major experimental group, abbreviated as US-CaCO₃:Ce or US-CaCO₃:Eu.

The body weight, body temperature, weight and waistline of the experimental rats were measured and recorded every week. At the end of the experiment, the rats were sacrificed and the blood was collected directly from the heart. The subcutaneous fat and organs were harvested for further analysis.

1.8.2 Serological and Blood Elements Analysis

In the serum analysis, the blood was collected in a blood collection tube (450533, Greiner bio-one, Austria), and centrifuged at 3500 rpm for 10 minutes in a centrifuge (5500, Kubota, Japan). The supernatant was collected and analyzed. Blood lipid (TC, TG), liver function (AST, ALT), renal function (BUN, Creatinine, UA), and calcium (Ca) were analyzed by serology analyzer (DRI/CHEN NX-500 I, Fuji, Japan).

In the blood elements, the blood was collected in a purple collection tube containing an EDTA anticoagulant, and mixed homogeneously for analysis. The number of white blood cells (WBC), red blood cells (RBC), hemoglobin (HGB), hematocrit ratio (HCT), platelets (PLT), neutrophil (NE), eosinophilic multinuclear (EO), basophil (BA), lymphocytes (LY), and mononuclear spheres (MO) were analyzed by hematology analyzer (BC-5000 VET, Mindray, China).

The two analysis were to check the safety of the new developed lipolysis method on the experimental animal. The results were recorded and summarized in the supplementary data.

1.8.3 Histological Sectioning with Hematoxylin and Eosin (H&E) Stain

The tissue sample of heart, liver, spleen, lungs, and kidneys were harvested by a sterilized surgical instrument. The tissues were carefully trimmed the surroundings and cleaned by PBS; and then placed in a 10% formalin solution (HT501128, Sigma, USA) for fixation. It was then immersed in acetone to de-oil and dehydrated by series of alcohol from 70% to 100%. The tissue was paraffin embedding in a tissue embedder (TEC-6, Tissue-Tek, USA). The paraffin blocks were sectioned (5 mm thick sections) on a rotary microtome (RM 215, Leica, Germany), and then the sections were fixed in 4% paraformaldehyde for 20 minutes, and washed 2 times by ddH₂O for 30 seconds. Dipped the slides into a Coplin jar containing hematoxylin solution for 30 seconds. Rinsed slides by ddH₂O for 1 minute, and then stained with 1% eosin Y solution for 20 seconds. Dehydrated the sections with 2 times by 95% alcohol and 2 changed of 100% alcohol. The sections were cleaned by xylene for 5 minutes and put on cover slide by mounting media (Zihayat et al., 2018). The images were observation by an optical microscope (Eclipse 80i, Nikon, Japan). The results were summarized in the supplementary data.

1.9 Statistic Method

All the experiments were conducted at least in triplicate, and the data was presented with means±standard deviation. Statistical analyses were performed by one-way ANOVA. The results were considered significant difference when the p-value<0.05.

2. Results

2.1 Material Characterization

2.1.1 the Crystal Structure Identification

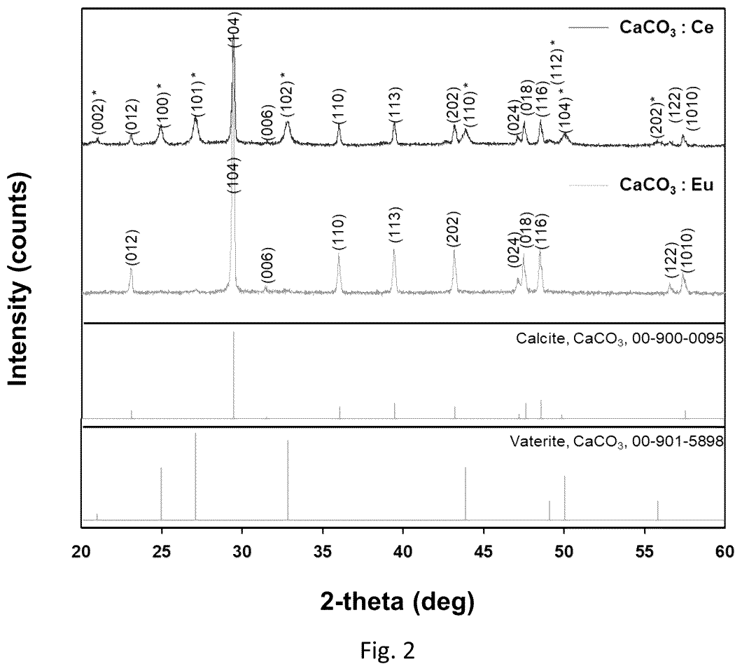

FIG. 2 showed XRD patterns of the synthesized CaCO₃:Ce or CaCO₃:Eu. The peaks and relative intensities of the synthesized CaCO₃:Ce or CaCO₃:Eu were fully matched to the calcite CaCO₃ as Crystallography Open Database (COD) No. 00-900-0095 and No. 00-901-5898, respectively.

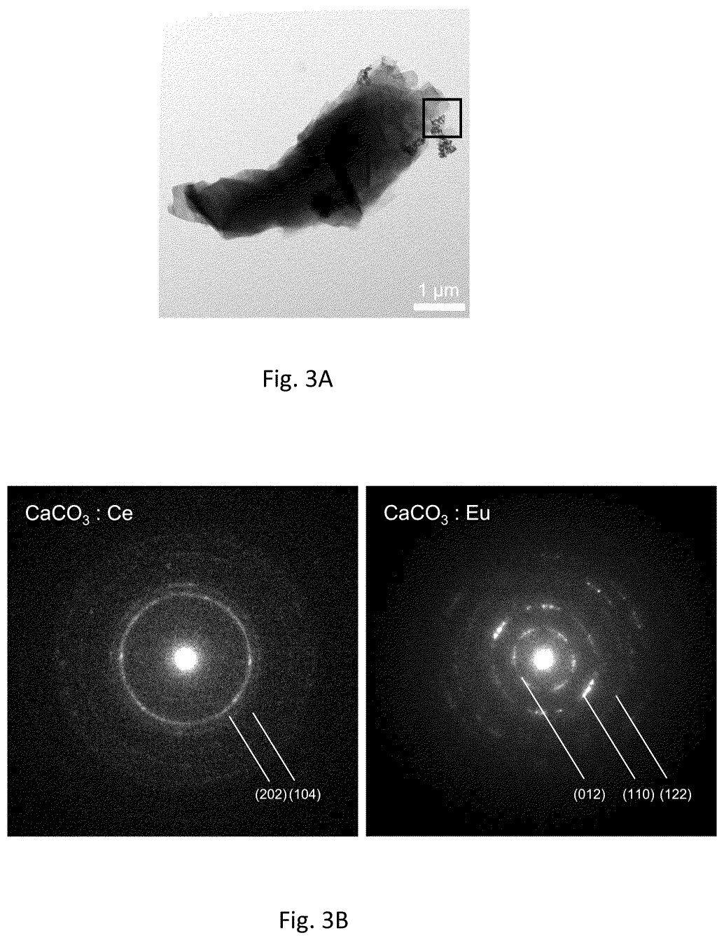

The synthesized CaCO₃:Eu further examined under the TEM; that showed a “nailhead” or “dogtooth” spar of calcite crystals that grew and aggregated with different habits, as shown in the edge in upper right of the FIG. 3A. The selected electronic diffraction pattern (FIG. 3B) was a classic ring pattern; with which the d-spacings calculated from the ring pattern were in agreement with the plane of (012), (110), and (122) in calcite crystal structure. CaCO₃:Ce (202), (104)

2.1.2 the Morphological Examination and Grain Size Evaluation Under SEM

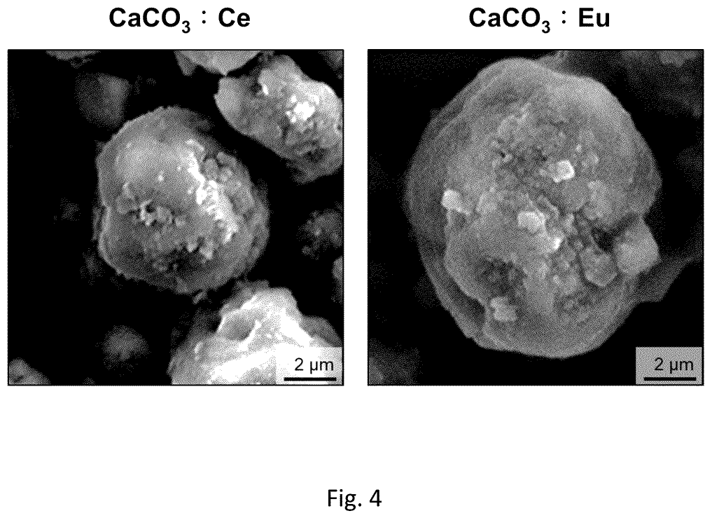

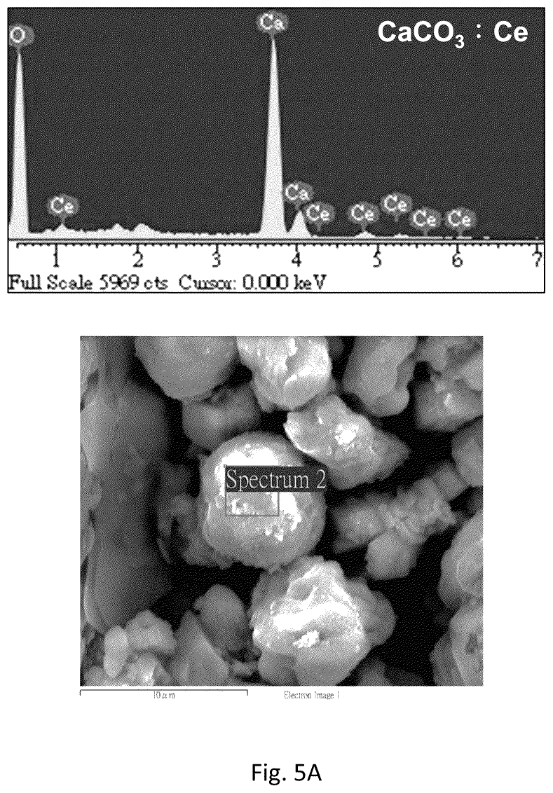

The surface morphologies of the developed CaCO₃:Ce and CaCO₃:Eu were examined under SEM as shown in FIG. 4. It was aggregated into a particle approximately 4 μm in average; that was composed by many small rhombohedral grains stacking into a particle. The particle was shaped as scalenohedron or prism by the nano-sized grains; that could be seen from the edge of TEM photo as FIG. 3A.

2.1.3 Chemical Composition Analysis

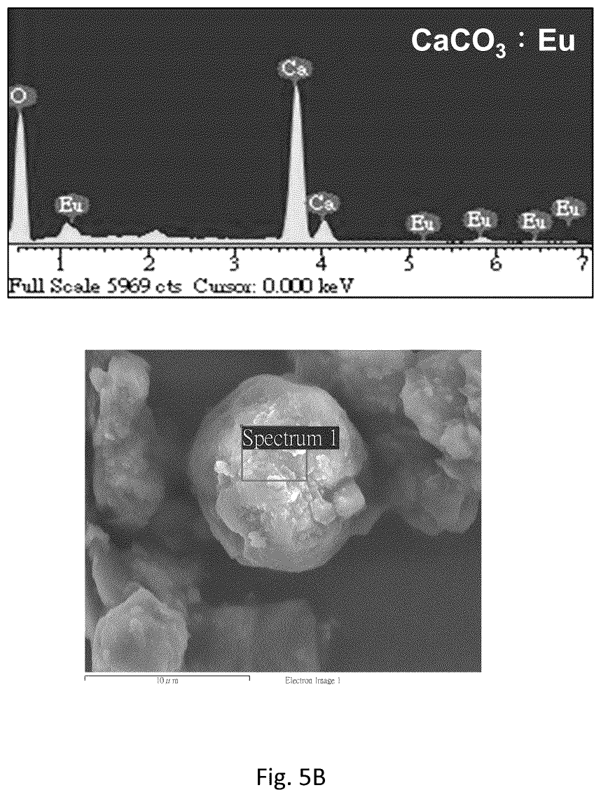

The overall elements composed in the synthesized CaCO₃:Ce and CaCO₃:Eu were detected by energy dispersed spectrophotometry to analyze the energy status of the electrons in different orbits as shown in FIGS. 5A and 5B, respectively; where the major elements were carbon, oxygen, calcium and cerium/europium. The average weight percentage (weight %) and average atomic percentage (atomic %) of each element for CaCO₃:Eu were shown in Tables 1a and 1b.

TABLE 1a
The average weight percentage (weight %) and
average atomic percentage (atomic %) of each element
of the synthesized CaCO₃:Eu.

| | Weight (%) | Atomic (%) |
|---|---|---|
| C K | 8.66 | 13.59 |
| O K | 63.79 | 75.15 |
| Ca K | 22.65 | 10.65 |
| Eu L | 4.90 | 0.61 |
| Totals | 100 | 100 |

TABLE 1b
The average weight percentage (weight %) and
average atomic percentage (atomic %) of each element
of the synthesized CaCO₃:Ce.

| | Weight (%) | Atomic (%) |
|---|---|---|
| C K | 11.06 | 17.34 |
| O K | 59.96 | 70.59 |
| Ca K | 24.34 | 11.44 |
| Ce L | 4.64 | 0.62 |
| Totals | 100 | 100 |

An ICP-MS was used to further confirm the concentration of Eu in synthesized particle. The concentration of Eu in CaCO₃:Eu was 112.5 mg/g (Tables 2a and 2b).

TABLE 2a
Measurement of Eu concentration in CaCO₃—Eu by ICP-MS.

| Sample | Eu (mg/g) |
|---|---|
| CaCO₃ | ND |
| CaCO₃:Eu | 112.5 |

TABLE 2b
Measurement of Eu concentration in CaCO₃—Ce by ICP-MS.

| Sample | Ce (mg/g) |
|---|---|
| CaCO₃ | ND |
| CaCO₃:Ce | 137.4 |

2.1.4 the Analysis of Particle Size Distribution

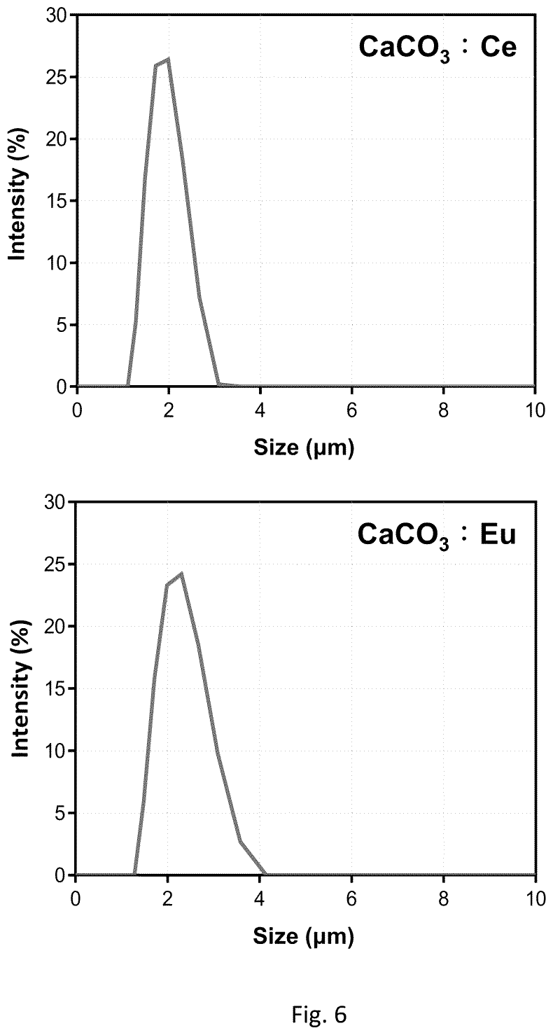

A Zeta-sizer was used to analyze the particle size and distribution of the synthesized CaCO₃:Ce and CaCO₃:Eu. As shown in FIG. 6, the particle size of CaCO₃:Eu was approximately 2.1 μm in average, and the size distribution of CaCO₃:Eu is from 1.48 to 3.58 μm; that was very close to the 4 μm in average observed under SEM. The size distribution of CaCO₃:Ce is 1.7 μm in average (1.28-3.09 μm). In the SEM picture (FIG. 4), the particle might more aggregate into a bigger one during drying process in the sample preparation. We believe that the developed CaCO₃:Ce and CaCO₃:Eu with adequate particle size could be uptake by the defense cells, such as phagocyte, macrophage etc., for the later-on controlled release by endosome-lysosome complex breaking down and pumping out to extra-cellular matrix, finally delivered to whole body by the surrounding capillary system. We would prove it in the later experiments.

2.2 Evaluation of Cytotoxicity In-Vitro

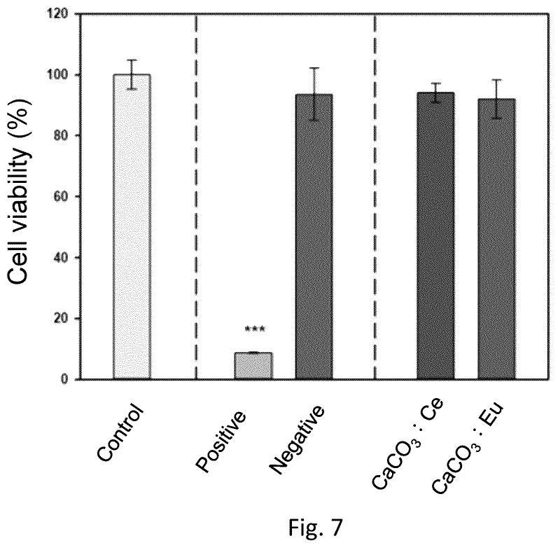

FIG. 7 showed the cell viability of the developed CaCO₃:Ce and CaCO₃:Eu followed the guideline of ISO 10993-5. The cell viability of the control group, P-control, N-control and experimental group of CaCO₃:Eu were 100±4.82, 8.70±0.19, 93.57±8.54, and 92.05±6.293, respectively. The difference of OD value between control group and CaCO₃:Eu was less than 25%. We could tell that the synthesized CaCO₃:Eu would not induce cytotoxicity to L-929 cells; and would keep cellular metabolism and mitochondrial functions in normal. The cell viability of experimental group of CaCO₃:Ce was 94.08±3.23. Therefore, the rare-earth element doped calcium carbonate particles have good biocompatibility.

2.3 ROS Generation of Synthesized Particles Expose to Ultrasonic Irradiation

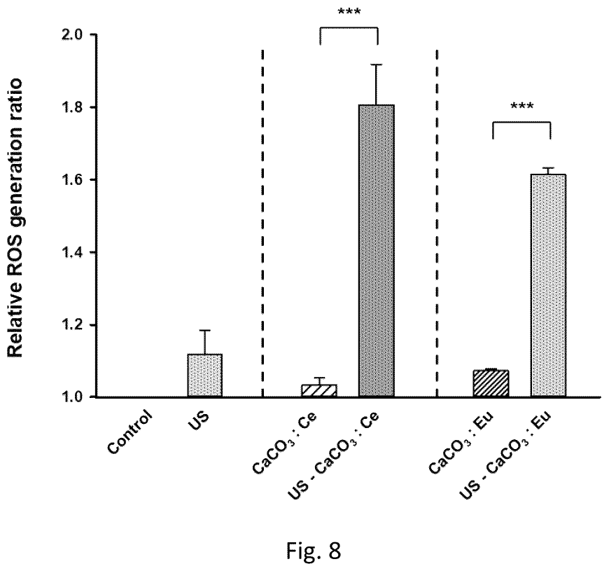

Intracellular ROS production was measured by a staining kit of CM-H₂DCFDA. The average fluorescence intensity of the control group was normalized as 1; the value of the other groups was normalized based on the intensity of control group as the relative value. The relative value would be in terms of the relative ROS production. After 3T3-L1 cells uptake the developed particles and then exposed to ultrasonic irradiation, the relative ROS production was determined and shown in FIG. 8. The ultrasound only group (Ultrasound, US) produced only 8% ROS relative to the control group (Control). In the material only groups, the ROS generation of CaCO₃:Ce was 23% and that of CaCO₃:Eu was 12%. In the group of combined treatment (sonosensitizer plus sonication), the ROS generation of CaCO₃:Ce reached 73% and that of CaCO₃:Eu is reached 69%.

We could see that the 3T3-L1 treated separately only by ultrasound irradiation (US) and only by synthesized particles (CaCO₃:Ce or CaCO₃:Eu) would induce only small amount of ROS production; whereas the cells treated the combination of ultrasonic irradiation and the synthesized CaCO₃:Ce (US-CaCO₃:Ce) or the combination of ultrasonic irradiation and CaCO₃:Eu (US-CaCO₃:Eu) would induce great amount ROS generation.

From the results, we could tell that the developed rare-earth element doped calcium carbonate particles would be a good sonosensitizer to generate energy under the excitation of ultrasound irradiation to produce ROS for lipolysis application.

2.4 the Efficacy of Synthesized Particles Exposed to Ultrasound Stimulation to Induce Adipocyte Necrosis Under ROS Stress

The efficacy of synthesized particles exposed to ultrasound stimulation to induce adipocyte necrosis under ROS stress was evaluated by WST-1 assay and live/dead stain to check the mitochondria activity and cell death rate, respectively.

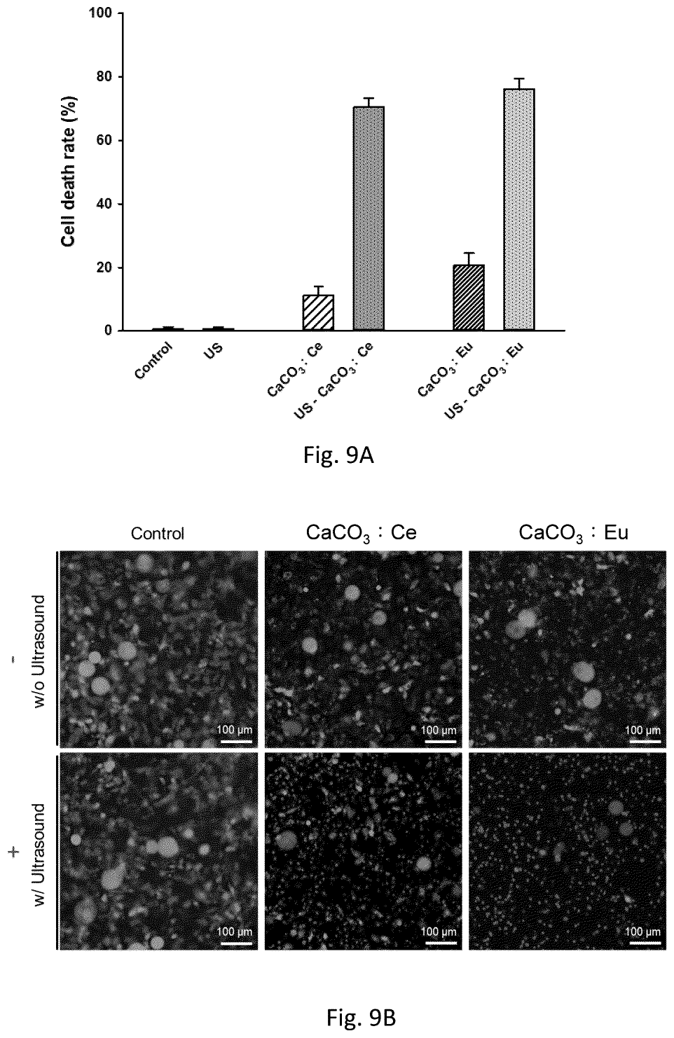

The cell viability is the same as the previous description to normalize the OD value to the control group as 1; and then the value in the other groups was normalized referred to the control group to obtain a relative value. In FIG. 9A, the adipocyte treated separately only by ultrasonic irradiation (US) and only by the developed particles (CaCO₃:Ce or CaCO₃:Eu) would keep the mitochondria in normal function as control group (Control). In the contrary, the mitochondria function or cell viability was far less than that of the control group for the cells treated the combination of ultrasound irradiation and the developed particles (US-CaCO₃:Ce or US-CaCO₃:Eu).

In the FIG. 9B, the death rate of the adipocyte evaluated by live/dead stain had the same results as the previous WST-1 test; where the cell in green and in red were representative to living and dead cells, respectively. The results showed that the cells treated with the combination of ultrasound and developed particles had the highest death rate of 75%, compared with the control group.

From the results of WST-1 and live/dead stain, we believe that the cells treated with the combination of ultrasound and developed particles could effectively generate ROS to make the adipocyte toward necrosis under the stress.

2.5 the Body Weight Growing Rate of the Rat Treated with CaCO₃:Eu and Exposed to Ultrasonic Irradiation

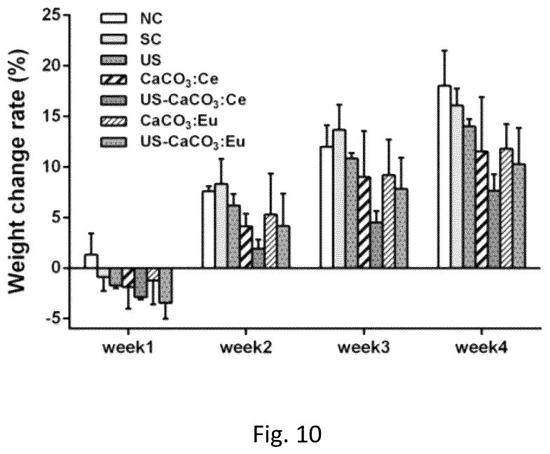

The FIG. 10 was the body weight growing rate of the rats injected with synthesized particles (CaCO₃:Ce or CaCO₃:Eu) to abdomen area and then applied with low-intensity ultrasound. The body weight growing rate of the rats without any treatment (Control) was much higher than the rats treated with the combination of CaCO₃:Ce or CaCO₃:Eu injection and low-intensity ultrasonic irradiation (US-CaCO₃:Ce or US-CaCO₃:Eu). The growth rate of control group was 7.57, 11.99 and 18.01 at week 2, 3 and 4, respectively. The growth rate of the group US-CaCO₃:Eu was 4.16, 7.83, and 10.67, respectively, at week 2, 3, and 4. US-CaCO₃:Ce was 1.89, 4.49, and 7.62, respectively, at week 2, 3, and 4. Weight change rate: (ending weight−starting weight)/starting weight×100%. During the experimental period, the animals' body weight did not change much between the groups at the beginning. With the passage of time, the combined treatment of sonosensitizers with ultrasound irradiation resulted in decreasing trend of the body weight at week 4. From the results of the experiment, the combination treatment could effectively inhibit growth rate on body weight.

2.6 Waistline Measurement

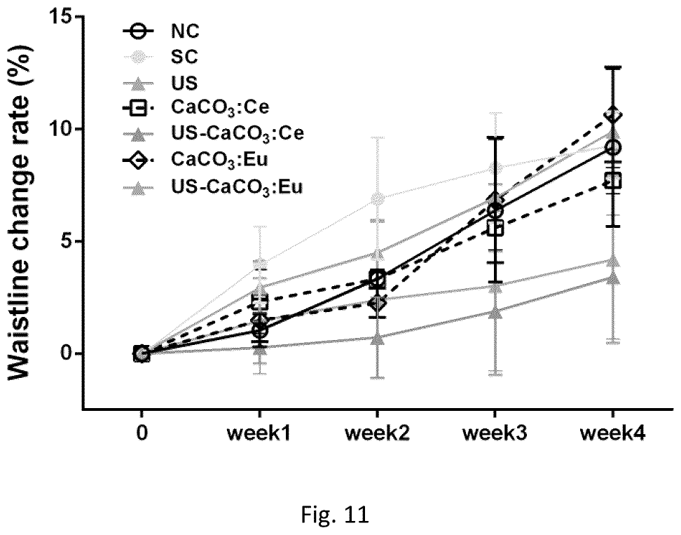

FIG. 11 was the waistline measurement of the experiment rats. The waistline of the rats treated with the combination of developed particle and low-power ultrasound was much lower than the control group and sham group. The waistline for the combination treatment (US-CaCO₃: Eu) was about 2.39, 3.02, and 4.19 at week 2, 3, and 4, respectively, while the waistline for the combination treatment (US-CaCO₃: Ce) was about 0.72, 1.88, and 3.40 at week 2, 3, and 4. The tendency was quite similar to the that of the body weight growth. From the results of the experiment, the combination treatment could effectively inhibit growth rate on waistline.

2.7 the Growth Rate of Subcutaneous Fat

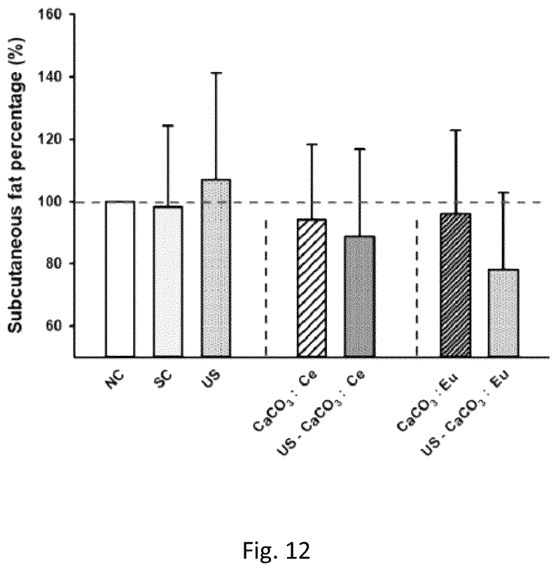

The abdominal subcutaneous fat percentage was calculated as subcutaneous fat mass around abdomen, relative to total body mass (subcutaneous fat mass/body weight×100%). The subcutaneous fat percentage was as shown in FIG. 12. The subcutaneous fat percentage for the rats treated with the combination of the CaCO₃:Ce or CaCO₃:Eu injection and ultrasound stimulation at week 4 was 88.7% and 78.3, respectively, compared with control group as 100%.

From the results of the experiment, the combination treatment could effectively inhibit growth rate on subcutaneous fat.

CaCO₃, comprises more than 4% of the mineral on earth's crust and is found throughout the world. Its most common natural forms are chalk, limestone, and marble, produced by the sedimentation of the shells of small fossilized snails, shellfish, and coral over millions of years (Mar and Phyo, 2013; Castro-Alonso et al., 2019). CaCO₃ has been widely used in medical applications, such as bone graft for tissue repair, biodegradable vehicle for drug and gene delivery etc. (Maleki et al., 2015; Song et al., 2018). In this study, we used Eu-doped calcium carbonate as sonodynamic reagent to combine with ultrasonic irradiation for body sculpture. Eu is a non-toxic rare earth element with an atomic number of 63, which belongs to the trivalent ion (Li et al., 2020). Eu could replace the calcium ion position of calcium carbonate to promote defects in calcium carbonate and increase the number of electron-hole pairs. Compared to divalent Ca ions, the doped Eu ions can obtain additional electrons, which creates a new energy level near the conduction band to reduce the energy gap effectively (Han et al., 2014; Wang et al., 2014). This makes the sonosensitizer more susceptible to ultrasonic irradiation and stimulates the generation of singlet oxygen and ROS in adipocytes for increasing the effective on lipolysis.

The CaCO₃:Eu was successfully synthesized using the eco-friendly method. The crystal structure was identified by XRD, which was matched with the standard pattern of calcite CaCO₃ (FIG. 2). Zeta-sizer was used to analyze the particle size and distribution of the synthesized particles. The average particle size of CaCO₃:Eu was 2.1 μm, which fall in the range of optimum particle size for cellular endocytosis (0.5-10 μm) (Hirota and Ter, 2012; Foroozandeh and Aziz, 2018). The particle size was further evaluated by TEM and SEM, those supposedly larger than that of Zeta-sizer due to the aggregation during the sample preparation before examined under electronic microscope. The real grain size was around 100-300 nm as shown in the electron-penetrated edge of the TEM picture (FIG. 3A).

Sonosensitizers can be divided into organic-based compounds and inorganic-based particles (Rosenthal et al., 2004; Chen et al., 2014). The organic-based materials, such as porphyrin-based structures, were reported to have short life span under ultrasound irradiation and showed great cytotoxicity. The inorganic-based particles, such as Ag, Au, Pt, TiO₂, and quantum dots, etc., have been used as sonosensitizer on sonodynamic therapy (SDT) for tumor/cancer treatment with better biostability and much longer life span (Xu et al., 2016). However, this kinds of material produce too much of ROS after exposed to ultrasonic stimulation, that is too strong and may result to the higher cytotoxicity (Serpe et al., 2012). In addition, the inorganic-based materials are not biodegradable in the human body. In the study, we develop a mild sonosensitizer CaCO₃:Eu for body sculpture after exposed to ultrasound. It is a biodegradable particle that can be decomposed in endosome-lysosome complex, and then turn into carbon dioxide (CO₂) and calcium ions (Ca²⁺), as described in the following series of reactions (Yang et al., 2019).

CaCO₃₍ₛ₎→Ca²⁺+CO₃²⁻ (1)

CO₃²⁻+H⁺→HCO³⁻ (2)

HCO³⁻+H⁺→H₂CO₃ (3)

H₂CO₃→H₂O+CO₂ (4)

where the CO₂ could serve as bomb to break down the endosome-lysosome complex and as one of mechanisms to kill the adipocytes in the fat tissue. The high concentration of Ca²⁺ ions, decomposed from the CaCO₃:Eu, could create a osmotic pressure to quickly escape from the complex environment. The adipose tissue with locally high level of Ca²⁺ ions would have the effect to the inhibition on the conversion of pre-adipocyte to adipocyte as following discussions.

Ca²⁺ ion has been investigated that was in association with adipocyte lipid metabolism, such as lipid synthesis and catabolism (Shapses et al., 2004; Duncan et al., 2007). Extracellular Ca²⁺ is also involved in the modulation of adipogenesis. It has been reported that high extracellular Ca²⁺ inhibits adipogenesis in 3T3-L1 pre-adipocytes (Jensen et al., 2004; Zhai et al., 2020). The process of pre-adipocyte differentiation of mature adipocytes is regulated by complex transcription factors, which can regulate the expression of hundreds of proteins responsible for establishing mature adipocyte phenotypes (Lowe et al., 2011). The two major adipogenic factors are peroxisome proliferator-activated receptor (PPARγ) and cytosine-cytosine-adenosine-adenosine-thymidine/enhancer-binding protein (C/EBP) (Farmer, 2006; Payne et al., 2009). Once the CaCO₃:Eu is decomposed by cells, calcium ions diffuse into the interstitial space, turning the entire local environment into a high-calcium environment. A high-calcium concentration in the microenvironment activates the preadipocyte factor 1 (PREF1) expression, which causes the up-regulation of the transcription factor SOX9 (Wang and Sul, 2009), that could inhibit the formation of sterol regulatory element-binding protein (SREBP), C/EBP, and PPARγ for pre-adipogenic cell maturation (Jensen et al., 2004; Vergara et al., 2016; Das and Choudhuri, 2017; Pramme-Steinwachs et al., 2017). In this study, we cultured the cells with different concentration of calcium ions in the cell culture medium, this result was verified that, 3T3-L1 cells under high-calcium ion environment were inhibited the differentiation of fat precursor cells into adipocytes (data not shown).

Sonoluminescence is a sonosensitizer absorb energy from inertial cavitation followed bubble rapture after ultrasound applied to the local tissue to produce ROS. The ROS include superoxide ions (O₂⁻), peroxide ions (O₂²⁻), hydroxyl radicals (OH), and singlet oxygen (¹O₂), which can cause to cell death in fat tissue (Kuroki et al., 2007; Trendowski, 2014; 2015; Pang et al., 2016). The CMH₂-DCFDA fluorescent dye was used to detect hydroxyl, peroxyl, and other ROS-active oxides in the cells. In this study, ROS production in the US group was 1.12 times higher than that in the control group. It is speculated that under the action of ultrasound, the generation of inertial cavitation finally causes the bubble to rupture, it could release strong energy that causes pyrolysis of surrounding water molecules, and producing hydroxyl groups in adipocytes. The production of ROS in the CaCO₃:Eu group was not observed. In addition, compare with US-CaCO₃:Eu and bare CaCO₃ under ultrasound irradiation (US-CaCO₃) group, ROS production of US-CaCO₃:Eu was 1.24 times high than US-CaCO₃ (data not shown). Meanwhile, in the US-CaCO₃:Eu group, the ROS production is 1.61 times higher than control, which is presumed to be inertial cavitation and the generation of sonoluminescence, causing the acoustic-sensitive materials to be excited and produce singlet oxygen and superoxide. The results show that the combination of CaCO₃:Eu and ultrasound treatment could produce more ROS free radicals on adipocytes. In addition, we also used the WST-1 and live/dead assays to verify the in vitro carving effect of CaCO₃:Eu under ultrasound irradiation. The US group showed that only inertial cavitation acts on the pyrolysis of water molecules to produce hydroxyl, which has limited oxidative damage capacity in adipocyte. When ultrasound is applied to activate CaCO₃:Eu, inertial cavitation pyrolysis produces hydroxyl and sonoluminescence excitation material, causing sonosensitizers to be excited to produce singlet oxygen and ROS. These results indicated that combination of CaCO₃:Eu and ultrasound treatment could cause significant damage to the adipocyte.

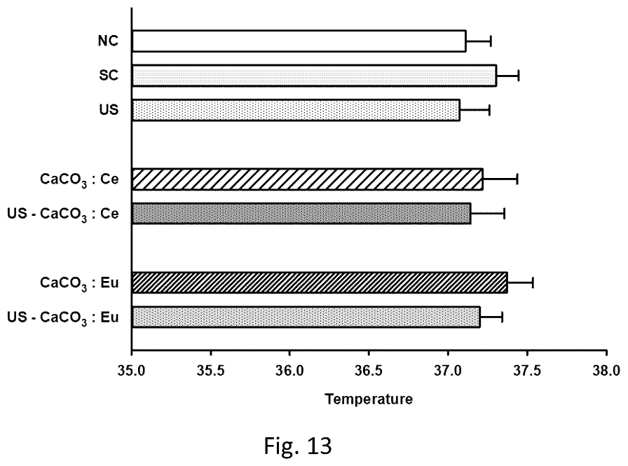

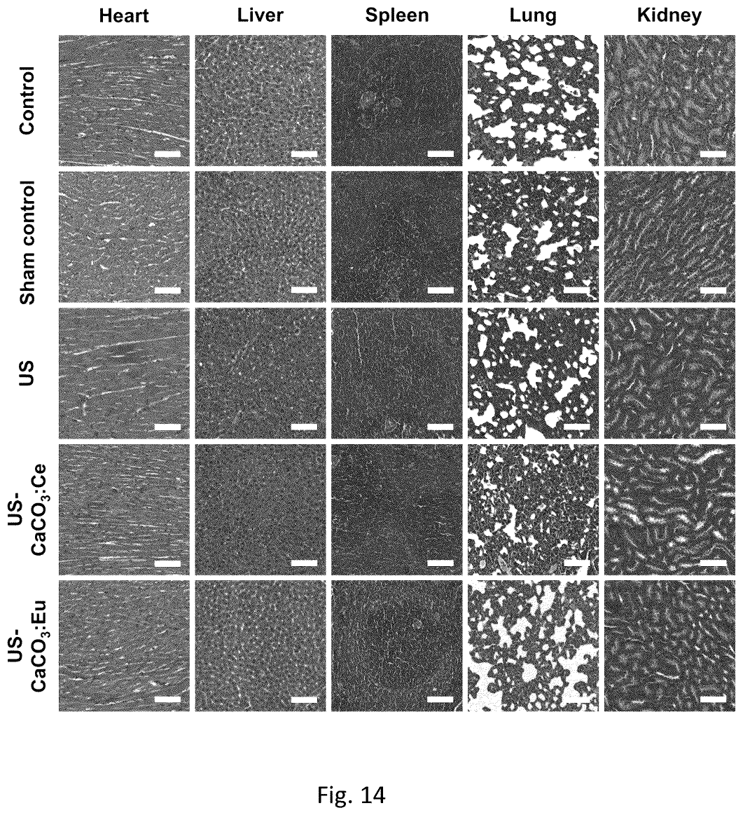

The results of animal study did not remarkably change between the groups at the beginning. Nevertheless, at a specific time, the US-CaCO₃:Eu sonodynamic treatment groups had a change in waistline within 4 weeks, and a statistical difference was reached in the fourth week. As the animal model used in this study was Sprague Dawley rats, the abdominal viscera and muscle tissue were removed, and the subcutaneous fat was measured based on the actual waist circumference. The results indicated the US-CaCO₃:Eu on SD rats could significantly decrease the growth rate of body weight and waistline and reduce the storage of adipose tissue by the weight of subcutaneous fats. In addition, the reduction in subcutaneous fat cell volume was observed from fat tissue section between Control and US-CaCO₃:Eu group (data not shown). Body temperature changes (FIG. 13), tissue sections (FIG. 14), and blood analysis (Table 3) of the above animal experiments showed that the injection of acoustically sensitive materials in animals and the effects of ultrasound of the rats are safe, and does not affect the physiological condition and organs of the rats by the ultrasound effect. The CaCO₃:Eu exposed to ultrasound irradiation on SD rats could significantly decrease body weight, waistline, and subcutaneous adipose tissue.

TABLE 3
Biochemical and hematological tests.

| Variables | NC | US-CaCO₃:Eu |
|---|---|---|
| WBC (K/μL) | 9.3 ± 2.9 | 7.3 ± 2.6 |
| NE (%) | 23.1 ± 8.8 | 18.1 ± 10.6 |
| LY (%) | 68.5 ± 10.7 | 76.2 ± 13.9 |
| MO (%) | 4.9 ± 0.5 | 4.0 ± 1.6 |
| EO (%) | 2.2 ± 1.0 | 1.0 ± 1.4 |
| BA (%) | 1.4 ± 0.39 | 0.7 ± 1.1 |
| RBC (M/μL) | 7.8 ± 1.4 | 7.8 ± 0.5 |
| HGB (g/dL) | 15.66 ± 0.6 | 13.9 ± 1.8 |
| HCT (%) | 49.4 ± 7.8 | 43.8 ± 3.8 |
| PLT (K/μL) | 976 ± 313 | 981 ± 119 |
| AST (U/L) | 121 ± 6 | 143 ± 26 |
| ALT (U/L) | 39.0 ± 9.2 | 40.7 ± 11.9 |
| BUN (mg/dL) | 20.2 ± 3.0 | 18.6 ± 3.3 |
| CRE (mg/dL) | 0.4 ± 0.2 | 0.2 ± 0.0 |
| UA (mg/dL) | 1.5 ± 0.2 | 1.2 ± 0.5 |
| TG (mg/dL) | 117 ± 29 | 142 ± 6 |
| TC (mg/dL) | 111 ± 1 | 73 ± 12 |
| Ca (mg/dL) | 11.0 ± 0.5 | 9.3 ± 0.6 |

WBC—White blood cell; NE—Neutrophil; LY—Lymphocyte; MO—Monocyte; EO—Eosinophil; BA—Basophil; RBC—Red blood cell; HGB—Hemoglobin; HCT—Hematocrit; PLT—Platelet; AST—Aspartate aminotransferase; ALT—Alanine aminotransferase; BUN—Blood urea nitrogen; CRE—Creatinine; UA—Uric acid; TG—Triglycerides; TC—Total cholesterol; Ca—Calcium.

In summary, the US-CaCO₃:Eu sonodynamic treatment is demonstrated that has a great potential in the application of body sculpture.

In the study, a sonosensitizer of rare-earth element doped calcium carbonate particles was successfully synthesized to combine with low-intensity ultrasound for body sculpture. The results showed that the CaCO₃:Ce or CaCO₃:Eu had good biocompatibility and could produce ROS in adipocytes for lipolysis. In addition, the results showed that developed sonosensitizer could effectively inhibit the adipogenesis after treated with low-intensity ultrasound. After 4-week animal study, the developed particles exposed to ultrasound irradiation on SD rats could significantly decrease the growth rate of body weight and waistline; and could reduce the storage of adipose tissue by the weight of subcutaneous fats. We could say that the combination of the developed rare-earth element doped calcium carbonate particles and low-intensity ultrasound could effectively inhibit the adipogenesis without skin burning and charred sounding tissue; that would be a mild and non-invasive treatment for the body sculpture.

### REFERENCES

Cao, Z., Yang, H., Kong, L., Gu, D., He, Z., Xu, Z., et al. (2012). Growth arrest induction of 3T3-L1 preadipocytes by serum starvation and their differentiation by the hormonal adipogenic cocktail. Journal of Cell and Animal Biology 6(5), 57-65.
Castro-Alonso, M. J., Montañez-Hernandez, L. E., Sanchez-Muñoz, M. A., Macias Franco, M. R., Narayanasamy, R., and Balagurusamy, N. (2019). Microbially Induced Calcium Carbonate Precipitation (MICP) and Its Potential in Bioconcrete: Microbiological and Molecular Concepts. Frontiers in Materials 6(126). doi: 10.3389/fmats.2019.00126.
Chen, H., Zhou, X., Gao, Y., Zheng, B., Tang, F., and Huang, J. (2014). Recent progress in development of new sonosensitizers for sonodynamic cancer therapy. Drug Discov Today 19, 502-509. doi: 10.1016/j.drudis.2014.01.010.
Das, S., and Choudhuri, D. (2017). Role of Low Calcium and High Calcium Diet on Adipocyte Metabolism with Respect to Serum Parathyroid Hormone (PTH) Levels in Male Wistar Rats. Indian journal of physiology and pharmacology 61, 430-439.
de Gusmao, P. R., Canella, C., de Gusmao, B. R., Filippo, A. A., and Filippo, G. R. (2020). Cryolipolysis for local fat reduction in adults from Brazil: A single-arm intervention study. J Cosmet Dermatol 19(11), 2898-2905. doi: 10.1111/jocd.13389.
Duncan, R. E., Ahmadian, M., Jaworski, K., Sarkadi-Nagy, E., and Sul, H. S. (2007). Regulation of lipolysis in adipocytes. Annual review of nutrition 27,79-101. doi: 10.1146/annurev.nutr.27.061406.093734.
Farmer, S. R. (2006). Transcriptional control of adipocyte formation. Cell Metab 4,263-273. doi: 10.1016/j.cmet.2006.07.001.
Foroozandeh, P., and Aziz, A. A. (2018). Insight into Cellular Uptake and Intracellular Trafficking of Nanoparticles. Nanoscale Res Lett 13(1), 339. doi: 10.1186/s11671-018-2728-6.
Han, Y. X., Yang, C. L., Wang, M. S., and Ma, X. G. (2014). Tuning the band gap and optical properties of γ-Si₃N₄ with rare earth element Ce. RSC Adv. 4, 55452-55458. doi: 10.1039/c4ra10519b.
Hirota, K., and Ter, H. (2012). Endocytosis of Particle Formulations by Macrophages and Its Application to Clinical Treatment. doi: 10.5772/45820.
Hsiao, M. Y., Lin, P. C., Lin, A. C., Wu, Y. W., Chen, W. S., and Lin, F. H. (2019). Oxidized hyaluronic acid/adipic acid dihydrazide hydrogel as drug-carrier for cytoprotective medications—preliminary results. Biomedical Engineering: Applications, Basis and Communications 31(05), 1950036.
Jalian, H. R., and Avram, M. M. (2012). Body contouring: the skinny on noninvasive fat removal. Semin Cutan Med Surg 31(2), 121-125. doi: 10.1016/j.sder.2012.02.004.
Jensen, B., Farach-Carson, M. C., Kenaley, E., and Akanbi, K. A. (2004). High extracellular calcium attenuates adipogenesis in 3T3-L1 preadipocytes. Exp Cell Res 301, 280-292. doi: 10.1016/j.yexcr.2004.08.030.
Jewell, M. L., Weiss, R. A., Baxter, R. A., Cox, S. E., Dover, J. S., Donofrio, L. M., et al. (2012). Safety and tolerability of high-intensity focused ultrasonography for noninvasive body sculpting: 24-week data from a randomized, sham-controlled study. Aesthet Surg J 32(7), 868-876. doi: 10.1177/1090820X12455190.
Jonnalagadda, U.S., Su, X., and Kwan, J. J. (2021). Nanostructured TiO₂ cavitation agents for dual-modal sonophotocatalysis with pulsed ultrasound. Ultrasonics Sonochemistry 73, 105530. doi: 10.1016/j.ultsonch.2021.105530.
Kordi, R., Dehghani, S., Noormohammadpour, P., Rostami, M., and Mansournia, M. A. (2015). Effect of abdominal resistance exercise on abdominal subcutaneous fat of obese women: a randomized controlled trial using ultrasound imaging assessments. J Manipulative Physiol Ther 38(3), 203-209. doi: 10.1016/j.jmpt.2014.12.004.
Kuroki, M., Hachimine, K., Abe, H., Shibaguchi, H., Kuroki, M., Maekawa, S.-i., et al. (2007). Sonodynamic therapy of cancer using novel sonosensitizers. 27, 3673-3677.
Li, S., Lu, X., Shi, S., Chen, L., Wang, Z., and Zhao, Y. (2020). Europium-Doped Ceria Nanowires as Anode for Solid Oxide Fuel Cells. Frontiers in Chemistry 8(348). doi: 10.3389/fchem.2020.00348.
Li, X., Yang, X., Liu, X., He, W., Huang, Q., Li, S., et al. (2018). Calcium carbonate nanoparticles promote osteogenesis compared to adipogenesis in human bone-marrow mesenchymal stem cells. Progress in Natural Science: Materials International 28(5), 598-608. doi: 10.1016/j.pnsc.2018.09.004.
Lowe, C. E., O'Rahilly, S., and Rochford, J. J. (2011). Adipogenesis at a glance. J Cell Sci 124, 2681-2686. doi: 10.1242/jcs.079699.
Maleki, S., Barzegar-Jalali, M., Zarrintan, M. H., Adibkia, K., and Lotfipour, F. (2015). Calcium carbonate nanoparticles; potential applications in bone and tooth disorders. Pharm Sci 20(4), 175-182.
Mar, A. A., and Phyo, H. M. (2013). Utilization of Calcium Carbonate (Clam Shell).
Mason, A. E., Jhaveri, K., Cohn, M., and Brewer, J. A. (2018). Testing a mobile mindful eating intervention targeting craving-related eating: feasibility and proof of concept. Journal of Behavioral Medicine 41(2), 160-173. doi: 10.1007/s10865-017-9884-5.
Michon, A. (2021). A Prospective Study Determining Patient Satisfaction with Combined Cryolipolysis and Shockwave Therapy Treatment for Noninvasive Body Contouring. Aesthetic Plast Surg. doi: 10.1007/s00266-021-02139-0.
Moreno-Moraga, J., Valero-Altes, T., Riquelme, A. M., Isarria-Marcosy, MI., and de la Torre, J. R. (2007). Body contouring by non-invasive transdermal focused ultrasound. Lasers Surg Med 39(4), 315-323. doi: 10.1002/lsm.20478.
Mrad, S., El Tawil, C., Sukaiti, W. A., Bou Chebl, R., Abou Dagher, G., and Kazzi, Z. (2019). Cardiac Arrest Following Liposuction: A Case Report of Lidocaine Toxicity. Oman Med J 34(4), 341-344. doi: 10.5001/omj.2019.66.
O'Neill, R. C., Hanson, S. E., Reece, E., and Winocour, S. (2021). Safety Considerations of Fat Grafting in Buttock Augmentation. Aesthetic Surgery Journal 41(Supplement_1), S25-S30. doi: 10.1093/asj/sjab092.
Pang, X., Xu, C., Jiang, Y., Xiao, Q., and Leung, A. W. (2016). Natural products in the discovery of novel sonosensitizers. Pharmacol Ther 162, 144-151. doi: 10.1016/j.pharmthera.2015.12.004.
Park, Y. K., Obiang-Obounou, B. W., Lee, J., Lee, T. Y., Bae, M. A., Hwang, K. S., et al. (2017). Anti-Adipogenic Effects on 3T3-L1 Cells and Zebrafish by Tanshinone IIA. Int J Mol Sci 18(10). doi: 10.3390/ijms18102065.
Payne, V. A., Au, W. S., Lowe, C. E., Rahman, S. M., Friedman, J. E., O'Rahilly, S., et al. (2009). C/EBP transcription factors regulate SREBP1c gene expression during adipogenesis. Biochem J 425, 215-223. doi: 10.1042/BJ20091112.
Pramme-Steinwachs, I., Jastroch, M., and Ussar, S. (2017). Extracellular calcium modulates brown adipocyte differentiation and identity. Sci Rep 7. doi: 10.1038/s41598-017-09025-3.
Rosenthal, I., Sostaric, J. Z., and Riesz, P. (2004). Sonodynamic therapy—a review of the synergistic effects of drugs and ultrasound. Ultrason Sonochem 11, 349-363. doi: 10.1016/j.ultsonch.2004.03.004.
Serpe, L., Foglietta, F., and Canaparo, R. (2012). Nanosonotechnology: the next challenge in cancer sonodynamic therapy. Nanotechnology Reviews 1. doi: 10.1515/ntrev-2011-0009.
Shapses, S. A., Heshka, S., and Heymsfield, S. B. (2004). Effect of calcium supplementation on weight and fat loss in women. The Journal of clinical endocrinology and metabolism 89(2), 632-637. doi: 10.1210/jc.2002-021136.
Shek, S. Y., Yeung, C. K., Chan, J. C., and Chan, H. H. (2014). Efficacy of high-intensity focused ultrasonography for noninvasive body sculpting in Chinese patients. Lasers Surg Med 46(4), 263-269. doi: 10.1002/lsm.22232.
Song, J., Wang, R., Liu, Z., and Zhang, H. (2018). Preparation and characterization of calcium carbonate microspheres and their potential application as drug carriers. Mol Med Rep 17(6), 8403-8408. doi: 10.3892/mmr.2018.8879.
Tonucci, L. B., Mourao, D. M., Ribeiro, A. Q., and Bressan, J. (2014). Noninvasive body contouring: biological and aesthetic effects of low-frequency, low-intensity ultrasound device. Aesthetic Plast Surg 38, 959-967. doi: 10.1007/s00266-014-0391-6.
Trendowski, M. (2014). The promise of sonodynamic therapy. Cancer Metastasis Rev 33, 143-160. doi: 10.1007/s10555-013-9461-5.
Trendowski, M. (2015). Using the Promise of Sonodynamic Therapy in the Clinical Setting against Disseminated Cancers. Chemother Res Pract. doi: 10.1155/2015/316015.
Vergara, E. J. S., Dela Cruz, J., Kim, C. M., and Hwang, S. G. (2016). Increased adipocyte differentiation may be mediated by extracellular calcium levels through effects on calreticulin and peroxisome proliferator activated receptor gamma expression in intramuscular stromal vascular cells isolated from Hanwoo beef cattle. Italian Journal of Animal Science 15, 256-263. doi: 10.1080/1828051x.2016.1186503.
Wang, H. Y., Gong, F. Z., Zhou, L. Y., and Sun, L. (2014). Facile Synthesis of CaCO₃ and CaCO₃:Eu³⁺ Phosphors by Solid State Reaction at Room Temperature and the Luminescence Properties. Materials Science Forum 809-810, 711-718. doi: 10.4028/www.scientific.net/MSF.809-810.711.
Wang, Y., and Sul, H. S. (2009). Pref-1 regulates mesenchymal cell commitment and differentiation through Sox9. Cell Metab 9, 287-302. doi: 10.1016/j.cmet.2009.01.013.
Witte, T., Dadras, M., Heck, F.-C., Heck, M., Habermalz, B., Welss, S., et al. (2020). Water-jet-assisted liposuction for the treatment of lipedema: Standardized treatment protocol and results of 63 patients. Journal of Plastic, Reconstructive & Aesthetic Surgery 73(9), 1637-1644. doi: 10.1016/j.bjps.2020.03.002.
Xiao, D., Cheng, J., Liang, W., Sun, L., and Zhao, J. (2021). Metal-phenolic coated and prochloraz-loaded calcium carbonate carriers with pH responsiveness for environmentally-safe fungicide delivery. Chemical Engineering Journal 418, 129274. doi: 10.1016/j.cej.2021.129274.
Xu, H., Zhang, X., Han, R., Yang, P., Ma, H., Song, Y., et al. (2016). Nanoparticles in sonodynamic therapy: state of the art review. RSC Advances 6(56), 50697-50705. doi: 10.1039/C6RA06862F.
Yang, C. C., Wang, C. X., Kuan, C. Y., Chi, C. Y., Chen, C. Y., Lin, Y. Y., et al. (2020). Using C-doped TiO2 Nanoparticles as a Novel Sonosensitizer for Cancer Treatment. Antioxidants 9(9), 880.
Yang, C. C., Wang, W. Y., Lin, F. H., and Hou, C. H. (2019). Rare-Earth-Doped Calcium Carbonate Exposed to X-ray Irradiation to Induce Reactive Oxygen Species for Tumor Treatment. Int J Mol Sci 20(5). doi: 10.3390/ijms20051148.
Zhai, M., Yang, D., Yi, W., and Sun, W. (2020). Involvement of calcium channels in the regulation of adipogenesis. Adipocyte 9(1), 132-141. doi: 10.1080/21623945.2020.1738792.
Zhou, B., Leung, B. Y. K., and Sun, L. (2017). The Effects of Low-Intensity Ultrasound on Fat Reduction of Rat Model. Biomed Res Int. doi: 10.1155/2017/4701481.
Zihayat, B., Khodadadi, A., Torabi, M., Mehdipour, M., Basin, M., and Asadi-Shekarri, M. (2018). Wound healing activity of sheep's bladder extracellular matrix in diabetic rats. Biomedical Engineering: Applications, Basis and Communications 30(02), 1850015. doi: 10.4015/S1016237218500151.

What is claimed is:

1. A method for reducing localized fat deposits in a subject in need thereof, comprising administering to a region with localized fat deposits of the subject a composition comprising an effective amount of rare-earth element doped calcium carbonate particles and applying to the region of the subject an ultrasonic irradiation having parameters so as to reduce the localized fat deposits in the region of the subject.

2. The method of claim 1, wherein the amount of the calcium carbonate particles is effective in increasing reactive oxygen species (ROS) production and producing carbon dioxide (CO₂) and calcium ions (Ca²⁺) in the region of administration under the ultrasonic irradiation.

3. The method of claim 1, wherein the amount of the calcium carbonate particles is effective in inducing adipocyte necrosis, inhibiting adipogenesis, and decreasing body weight under the ultrasonic irradiation.

4. The method of claim 1, wherein the region where calcium carbonate particles are administered to include back, shoulders, neck, chest, abdomen, thighs, hips, legs, arms and face.

5. The method of claim 1, wherein the calcium carbonate particles are administered through parenteral route.

6. The method of claim 1, wherein the rare-earth element is selected from the group consisting of lanthanum (La), cerium (Ce), praseodymium (Pr), neodymium (Nd), samarium (Sm), europium (Eu), gadolinium (Gd), terbium (Tb), dysprosium (Dy), holmium (Ho), erbium (Er), thulium (Tm), ytterbium (Yb), lutetium (Lu), yttrium (Y) and scandium (Sc).

7. The method of claim 1, wherein the rare-earth element is cerium (Ce) or europium (Eu).

8. The method of claim 7, wherein the calcium carbonate particles are aggregated to have a diameter in the range of 1 to 4 μm.

9. The method of claim 7, wherein the rare-earth element is europium (Eu).

10. The method of claim 1, wherein the ultrasonic irradiation has intensity of 0.1 to 10 W/cm².

11. The method of claim 10, wherein the ultrasonic irradiation has frequency of 0.1 to 10 MHz.

12. The method of claim 10, wherein the ultrasonic irradiation has a duty cycle ranging from 20% to 100%.

13. The method of claim 12, wherein the ultrasonic irradiation is applied 1 to 5 days per week, each day 5 to 600 seconds, for 1 to 6 weeks.

14. The method of claim 1, wherein the subject is overweight or obese.

15. The method of claim 1, wherein the subject is suffered from obesity.

16. The method of claim 1, wherein the subject has a BMI value of 18.5 to 24.9.

17. A method for reducing localized fat deposits in a subject in need thereof, comprising administering to a region with localized fat deposits of the subject a composition comprising an effective amount of cerium (Ce) or europium (Eu) doped calcium carbonate particles and applying to the region of the subject an ultrasonic irradiation having intensity of 0.1 to 10 W/cm², frequency of 0.1 to 10 MHz and a duty cycle ranging from 20% to 100%, for 1 to 5 days per week, each day for 5 to 600 seconds for 1 to 6 weeks.

18. The method of claim 17, wherein the region where calcium carbonate particles are administered to include back, shoulders, neck, chest, abdomen, thighs, hips, legs, arms and face.
